(19) United States
(12) Patent Application Publication
(10) Pub. No.: US 2023/0324346 A1
(43) Pub. Date: Oct. 12, 2023

(54) METHOD FOR MEASURING SPECIFIC MIGRATION AMOUNT OF ANTIOXIDANT IN PET/PE COMPOUND FOOD CONTACT MATERIAL

(71) Applicant: Technology Center of Nanning Customs, Nanning City (CN)

(72) Inventors: Xiangyu CAI, Nanning (CN); Yongfeng ZHAO, Nanning (CN); Lu CHEN, Nanning (CN); Chunqiu Lv, Nanning (CN)

(21) Appl. No.: 17/336,401

(22) Filed: Jun. 2, 2021

(30) Foreign Application Priority Data
Jul. 20, 2020 (CN) 2020106991639

(51) Int. Cl. G01N 30/06

(52) U.S. Cl. CPC G01N 30/06; G01N 2030/062; G01N 2030/027

(57) ABSTRACT

A method for measuring specific migration amount of an antioxidants in a polyethylene terephthalate (PET)/polyethylene (PE) compound food contact material is disclosed. More specifically, a method for simultaneously measuring the specific migration amount of 16 antioxidants in PET/PE compound food contact material while establishing a high performance liquid chromatography-tandem mass spectrometry (HPLC-MS/MS) is disclosed. The 16 target compounds determined by the method have a good linear relationship in the corresponding range, the correlation coefficient is greater than 0.995, the quantitative limit of the aqueous food simulant is 0.1-1.3 ng/kg, and the quantitative limit of the olive oil food simulant is 0.3-3.0 μg/kg. The average recovery is 81.0-112% and the relative standard deviation is 0.4-9.1% at the spiked level of 2.0-20 μg/kg. The disclosure has high sensitivity and low quantitative limit, and can meet the detection requirement of antioxidant specific migration in the PET/PE compound food contact material.

### FIELD OF THE INVENTION

The present disclosure relates to the technical field of analysis and detection, and more specifically, to a method for measuring specific migration amount of an antioxidant in a PET/PE compound food contact material.

### BACKGROUND ART

Currently, food packaging is mainly made of plastic. The common types of plastic packaging include polyethylene (PE), polypropylene (PP), polyethylene terephthalate (PET), polyvinyl chloride (PVC), polycarbonate (PC) and the like. Plastic products are easily oxidized and decomposed during production and use, and antioxidants are widely added to plastic articles due to the ability to effectively delay oxidation and decomposition of plastic products. The antioxidants added in plastic packaging are mainly synthetic antioxidants, and the toxicity of these substances is greater than that of natural antioxidants. If plastic products are used for packaging food products, these antioxidants and their decomposition products may migrate from the plastic product into the food product, thus endangering the health of consumers. The national standard GB 9685-2016 and European Commission Directive (EU) No 10/2011 both list the compounds that may migrate in plastic products and their specific migration into food or food simulants.

Currently, the detection method for antioxidant components in plastic food contact material products at home and abroad is mainly provided with gas chromatography (GC), gas chromatography tandem mass spectrometry (GC-MS), high performance liquid chromatography (HPLC), ultra-high performance liquid chromatography (UPLC), high performance liquid chromatography tandem mass spectrometry (LC-MS/MS), while existing methods exist for simultaneously detecting less total number of antioxidant components and higher quantitative limit. In the additive directory allowed in the national standard GB 9685-2016, there are also multiple antioxidant additives that have not been associated with the detection criteria.

Therefore, the development of a method for determining the amount of migration of various antioxidants in food simulants that can be used as a polyethylene terephthalate/polyethylene (PET/PE) compound food contact material has become an urgent problem to be solved by those skilled in the art.

### SUMMARY

The method can simultaneously determine 16 antioxidant components, wherein the antioxidant components comprise components in a national standard GB 9685-2016 and European Union Directive (EU) No 10/2011 permission list and components outside the permission list, provides a sensitive, accurate and rapid detection method for China food packaging market access, and provides technical guarantee for quality supervision of import and export food packaging material.

In order to achieve the purpose, the present disclosure provides the following technical solution:

A method for measuring specific migration amount of an antioxidant in a polyethylene terephthalate/polyethylene compound food contact material, wherein the specific steps comprising:

I. Standard Stock Solution Formulation

Weighing an antioxidant standard substance precisely and dissolving the antioxidant standard substance in methanol for constant volume to prepare a mixed standard stock solution with the concentration of 100 μg/mL, −20° C. for storage;

II. Mixed Standard Intermediate Stock Solution Formulation

Accurately sucking 1 mL of the mixed standard stock solution in the step I in a 100 mL volumetric flask respectively, and carrying out constant volume by methanol to obtain a mixed standard intermediate stock solution with the concentration of 1 μg/mL;

III. Aqueous Food Simulant Standard Working Solution Formulation

Transferring 5 μL, 10 μL, 20 μL, 50 μL, 100 μL, 200 μL of the mixed standard intermediate stock solution obtained in the step II into six 10-mL volumetric flasks respectively, carrying out volumetric calibration on the mixed standard intermediate stock solution by using an aqueous food simulant diluted by 10 times of methanol, and mixing uniformly to obtain a mixed standard working solution with the concentration of 0.5 ng/mL, 1.0 ng/mL, 2.0 ng/mL, 5.0 ng/mL, 10.0 ng/mL and 20.0 ng/mL to be tested;

IV. Lipid Food Simulant Standard Working Solution Formulation

Weighing 2 g olive oil into six 10 mL stoppered test tubes accurately, and adding 5 μL, 10 μL, 20 μL, 50 μL, 100 μL, 200 μL of the mixed standard intermediate stock of Step II to obtain a mixed standard working solution with the content of 0.5 ng/mL, 1.0 ng/mL, 2.0 ng/mL, 5.0 ng/mL, 10.0 ng/mL and 20.0 ng/mL, adding 10 mL of methanol into each test tube respectively, vortexing for 2 min, and standing for layering, then sucking the upper solution by a syringe, filtering through a 0.2 μm hydrophobic polytetrafluoroethylene filter membrane to be tested;

V. Migration Experiments

Referring to the migration test method and conditions of GB/T 23296.1-2009, selecting the simulant of aqueous food or lipid food to soak a sample;

VI. Sample Pretreatment Process

Diluting the aqueous food simulant with methanol for 10 times, mixing uniformly, and 1 mL of the diluted solution is aspirated by a glass syringe and filtered through a 0.22 μm PTFE syringe filter into a sample to be tested;

Weighing 2 g of olive oil food simulant into a 15 mL glass centrifuge tube with a plug, adding 5 mL of methanol, vortexing for 3 min, centrifuging for 5 min at 4000 r/min, removing the upper-layer methanol, repeatedly extracting the sample once with 5 mL of methanol, merging the upper-layer methanol, mixing uniformly, filtering into the sample through a 0.22 μm hydrophobic polytetrafluoroethylene needle filter, to be tested;

VII. Liquid Chromatography Conditions

Chromatographic column: shim-pack XR-ODS III (1.6 μm, 2.0 mm×75 nm), column temperature 40° C., mobile phase A water, mobile phase B methanol, flow rate 0.3 mL/min, injection volume 5 μL, elution gradient 0-8 min, 90% B-100% B; 8-12 min, 100% B; 12-13 min, 100% B-90% B; 13-15 min, 90% B;

VIII. Mass Spectrometry Conditions

An electrospray ion source, wherein the electrospray voltage is 5500V in a positive ion mode, 4500V in a negative ion mode, the atomization air pressure is 55 kPa, the air curtain air pressure is 35 kPa, the auxiliary air flow rate is 55 kPa, the ion source temperature is 600° C., the scanning mode is positive ion scanning and negative ion scanning, and the detection method is multi-reaction detection.

Preferably, the antioxidant standards in step I include one or more of Irganox DLTP, Irganox 425, Irganox 168, Irganox 405, Irganox 3114, Irganox 2246, Irganox 300, Irganox 697, Irganox CA, Irganox 245, Irganox 1290, Irganox 1024, Irganox CY, Irganox 1098, Irganox 1076, or BHA.

It should be noted that the 16 kinds of antioxidant components that are simultaneously measured in the present disclosure include both the National Standard GB 9685-2016 and the EU No. 10/2011 allow the components in the list and also contain ingredients that allow the outside of the list. The number, shorthand, name, CAS number, molecular formula, molecular weight, and SML limit of the 16 antioxidants are shown in Table 2, wherein each antioxidant component SML limit value is out of GB 9685-2016.

TABLE 2

| | | | CAS | Molecular | | SML/ |
|---|---|---|---|---|---|---|
| No. | Synonyms | Chemical name | No. | formula | Mw | (mg/kg) |
| 1 | Irganox | Dilauryl thiodipropionate | 123- | C₃₀H₅₈O₄S | 514.8 | — |
| | DLTP | | 28-4 | | | |
| 2 | Irganox | 2,2′-methylenebis[6- | 88-24- | C₂₅H₃₆O₂ | 368.5 | — |
| | 425 | (1,1-dimethylethyl)-4- | 4 | | | |
| | | ethyl-Phenol | | | | |
| 3 | Irganox | Tris(2,4-ditert-butyl- | 31570- | C₄₂H₆₃O₃P | 646.9 | — |
| | 168 | phenyl)phosphite | 04-4 | | | |
| 4 | Irganox | Bis[4-(2-phenyl-2- | 10081- | C₃₀H₃₁N | 405.5 | — |
| | 405 | propyl)phenyl]amine | 67-1 | | | |
| 5 | Irganox | Tris(3,5-di-tert-butyl-4- | 27676- | C₄₈H₆₉N₃O₆ | 784.0 | 5 |
| | 3114 | hydroxybenzyl) | 62-6 | | | |
| | | isocyanurate | | | | |
| 6 | Irganox | 2,2′-Methylene | 119- | C₂₃H₃₂O₂ | 340.4 | — |
| | 2246 | bis(6-tert-butyl-4- | 47-1 | | | |
| | | methylphenol) | | | | |
| 7 | Irganox | 4,4′-Thiobis(6-tert- | 96- | C₂₂H₃₀O₂S | 358.5 | 0.48 |
| | 300 | butyl-m-cresol) | 69-5 | | | |
| 8 | Irganox | (1,2-Dioxoethylene)bis | 70331- | C₄₀H₆0N₂O₈ | 696.9 | — |
| | 697 | (iminoethylene) | 94-1 | | | |
| | | bis(3-(3,5-di-tert-butyl-4- | | | | |
| | | hydroxyphenyl)propionate) | | | | |
| 9 | Irganox | 1,1,3-Tris(2-methyl-4- | 1843- | C₃₇H₅₂O₃ | 544.8 | 5 |
| | CA | hydroxy-5-tert- | 03- | | | |
| | | butylphenyl)butane | 4 | | | |
| 10 | Irganox | Triethylene glycol | 36443- | C₃₄H₅₀O₈ | 586.7 | 9 |
| | 245 | bis(3-tert-butyl-4-hydroxy- | 68- | | | |
| | | 5-methylphenyl) | 2 | | | |
| | | propionate | | | | |
| 11 | Irganox | 2,2′-Ethylidenebis(4,6-di- | 35958- | C₃₀H₄₆O₂ | 438.6 | 5 |
| | 1290 | tert-butylphenol) | 30-6 | | | |
| 12 | Irganox | 1,2-Bis(3,5-di-tert-butyl- | 32687- | C₃₄H₅₂N₂O₄ | 552.7 | 15 |
| | 1024 | 4-hydroxy- | 78-8 | | | |
| | | hydrocinnamoyl)hydrazine | | | | |
| 13 | Irganox | Tris(4-tert-butyl-3-hydroxy- | 40601- | C₄₂H₅₇N₃O₆ | 699.9 | 6 |
| | CY | 2,6-dimethylbenzyl) | 76-1 | | | |
| | | isocyanurate | | | | |
| 14 | Irganox | 3,3′-Bis(3,5-di-tert-butyl- | 23128- | C₄₀H₆₄N₂O₄ | 636.9 | 45 |
| | 1098 | 4-hydroxyphenyl)-N,N′- | 74- | | | |
| | | hexamethylene- | 7 | | | |
| | | dipropionamide | | | | |
| 15 | Irganox | Octadecyl | 2082- | C₃₅H₆₂O₃ | 530.8 | 6 |
| | 1076 | 3-(3,5-di-tert-butyl-4- | 79-3 | | | |
| | | hydroxyphenyl)propionate | | | | |
| 16 | BHA | Butylated hydroxyanisole | 25013- | C₁₁H₁₆O₂ | 180.2 | 30 |
| | | | 16-5 | | | |

Further preferably, the mass spectrometry condition further comprises a collision voltage CE of each antioxidant, a cluster voltage DP and a collision chamber outlet voltage CXP, the collision voltage CE of each antioxidant, the cluster voltage DP, and the collision chamber outlet voltage CXP are shown in Table 1.

TABLE 1

| Com- | | | | | | |
|---|---|---|---|---|---|---|
| pound | Com- | Ion pair | | DP | CE | CXP |
| No. | pound | (m/z) | Polarity | (eV) | (eV) | (eV) |
| 1 | Irganox | 515.4 > 143.1* | Positive | 200 | 23 | 16 |
| | DLTP | 515.4 > 115.0 | | | 35 | 12 |
| 2 | Irganox | 386.3 > 191.1* | Positive | 60 | 23 | 17 |
| | 425 | 386.3 > 257.1 | | | 15 | 37 |
| 3 | Irganox | 647.5 > 347.4* | Positive | 120 | 47 | 13 |
| | 168 | 647.5 > 147.1 | | | 47 | 13 |
| 4 | Irganox | 406.3 > 196.2* | Positive | 100 | 48 | 12 |
| | 405 | 406.3 > 119.1 | | | 41 | 11 |
| 5 | Irganox | 806.3 > 219.0* | Positive | 100 | 72 | 13 |
| | 3114 | 806.3 > 370.3 | | | 53 | 24 |
| 6 | Irganox | 358.2 > 177.1* | Positive | 50 | 22 | 10 |
| | 2246 | 358.2 > 229.2 | | | 13 | 16 |
| 7 | Irganox | 357.1 > 194.1* | Negative | −100 | −39 | −19 |
| | 300 | 357.1 > 179.2 | | | −59 | −11 |
| 8 | Irganox | 695.5 > 277.1* | Negative | −200 | −46 | −8 |
| | 697 | 695.5 > 417.2 | | | −34 | −33 |
| 9 | Irganox | 543.4 > 337.1* | Negative | −230 | −62 | −14 |
| | CA | 543.4 > 281.1 | | | −54 | −23 |
| 10 | Irganox | 585.4 > 367.1* | Negative | −100 | −39 | −23 |
| | 245 | 585.4 > 409.1 | | | −36 | −28 |
| 11 | Irganox | 438.3 > 205.2* | Negative | −50 | −42 | −10 |
| | 1290 | 438.3 > 232.2 | | | −36 | −20 |
| 12 | Irganox | 551.4 > 333.4* | Negative | −200 | −38 | −21 |
| | 1024 | 551.4 > 115.1 | | | −42 | −6 |
| 13 | Irganox | 698.5 > 508.2* | Negative | −100 | −38 | −13 |
| | CY | 698.5 > 232.0 | | | −58 | −18 |
| 14 | Irganox | 635.6 > 417.2* | Negative | −100 | −52 | −20 |
| | 1098 | 635.6 > 199.1 | | | −57 | −14 |
| 15 | Irganox | 529.2 > 267.2* | Negative | −100 | −51 | −24 |
| | 1076 | 529.2 > 269.2 | | | −39 | −23 |
| 16 | BHA | 179.1 > 149.0* | Negative | −50 | −31 | −15 |
| | | 179.1 > 164.1 | | | −20 | −10 |

Preferably, the aqueous food analog comprises ultrapure water, 4% acetic acid, or 10% ethanol.

Compared with the prior art, the disclosure establishes a high performance liquid chromatography tandem mass spectrometry (LC-MS/MS) method for simultaneously measuring the migration amount of antioxidants in food simulants of polyethylene terephthalate (PET/PE) compound food contact materials. The method has the advantages that the sample pretreatment is simple, the chromatographic separation effect is good, the accuracy is high, the quantitative limit completely meets the limit requirement on the specific migration amount of the 16 antioxidants in GB 9685-2016, and the method can be widely applied to import and export supervision and product quality control of the specific migration amount of the 16 antioxidants in the PE/PET composite food contact material.

### BRIEF DESCRIPTION OF THE DRAWINGS

in order to explain the embodiments of the present disclosure or the technical solutions in the prior art more clearly, the following will briefly introduce the drawings that need to be used in the description of the embodiments or the prior art. Obviously, the drawings in the following description are only embodiments of the present disclosure. For those of ordinary skill in the art, other drawings can be obtained according to the provided drawings without creative work.

FIG. 1 is a view showing the extraction of an antioxidant in the olive oil food simulant extraction solvent comparison of embodiment 1 of the present disclosure;

FIG. 2A is an extraction ion chromatogram of Irganox DLTP in a 10% ethanol food simulant;

FIG. 2B is an extraction ion chromatogram of Irganox 425 in a 10% ethanol food simulant;

FIG. 2C is an extraction ion chromatogram of Irganox 168 in a 10% ethanol food simulant;

FIG. 2D is an extraction ion chromatogram of Irganox 405 in a 10% ethanol food simulant;

FIG. 2E is an extraction ion chromatogram of Irganox 3114 in a 10% ethanol food simulant;

FIG. 2F is an extraction ion chromatogram of Irganox 2246 in a 10% ethanol food simulant;

FIG. 2G is an extraction ion chromatogram of Irganox 300 in a 10% ethanol food simulant;

FIG. 2H is an extraction ion chromatogram of Irganox 697 in a 10% ethanol food simulant;

FIG. 2I is an extraction ion chromatogram of Irganox CA in a 10% ethanol food simulant;

FIG. 2J is an extraction ion chromatogram of Irganox 245 in a 10% ethanol food simulant;

FIG. 2K is an extraction ion chromatogram of Irganox 1290 in a 10% ethanol food simulant;

FIG. 2L is an extraction ion chromatogram of Irganox 1024 in a 10% ethanol food simulant;

FIG. 2M is an extraction ion chromatogram of Irganox CY in a 10% ethanol food simulant;

FIG. 2N is an extraction ion chromatogram of Irganox 1098 in a 10% ethanol food simulant;

FIG. 2O is an extraction ion chromatogram of Irganox BHA in a 10% ethanol food simulant; and

FIG. 2P is an extraction ion chromatogram of Irganox 1076 in a 10% ethanol food simulant.

### DETAILED DESCRIPTION OF THE EMBODIMENTS

The embodiments of the present disclosure will be described in detail below. Examples of the embodiments are shown in the accompanying drawings, wherein the same or similar reference numerals indicate the same or similar elements or elements with the same or similar functions. The embodiments described below with reference to the drawings are exemplary, and are intended to explain the present disclosure, but should not be construed as limiting the present disclosure.

The disclosure discloses a method for simultaneously measuring the specific migration amount of 16 antioxidants in PET/PE compound food contact material while establishing high performance liquid chromatography-tandem mass spectrometry (HPLC-MS/MS). In this method, ultrapure water, 4% acetic acid, 10% ethanol and olive oil were used as food simulants to treat PET/PE composite food contact materials, using a Shim-pack XR-ODS III (1.6 μm, 2.0 mm×75 mm) chromatographic column with gradient elution, methanol and water were used as mobile phase gradient elution. Under the positive and negative ion mode, the electrospray ionization multi response monitoring (MRM) mode was used for qualitative and quantitative analysis, and external standard method was used for quantitative analysis. The method is high in sensitivity and low in quantitative limit, and meets the detection requirement of antioxidant specific migration in the PET/PE compound food contact material.

### Embodiment 1

A method for measuring specific migration amount of an antioxidant in a polyethylene terephthalate/polyethylene compound food contact material, wherein the specific steps comprising:

I. Standard Stock Solution Formulation

Weighing an antioxidant standard substance precisely and dissolving the antioxidant standard substance in methanol for constant volume to prepare a mixed standard stock solution with the concentration of 100 μg/mL, −20° C. for storage;

II. Mixed Standard Intermediate Stock Solution Formulation

Accurately sucking 1 mL of the mixed standard stock solution in the step I in a 100 mL volumetric flask respectively, and carrying out constant volume by methanol to obtain a mixed standard intermediate stock solution with the concentration of 1 s g/mL;

III. Aqueous Food Simulant Standard Working Solution Formulation

Transferring 5 μL, 10 μL, 20 μL, 50 μL, 100 μL, 200 μL of the mixed standard intermediate stock solution obtained in the step 11 into six 10-mL volumetric flasks respectively, carrying out volumetric calibration on the mixed standard intermediate stock solution by using an aqueous food simulant diluted by 10 times of methanol, and mixing uniformly to obtain a mixed standard working solution with the concentration of 0.5 ng/mL, 1.0 ng/mL, 2.0 ng/mL, 5.0 ng/mL, 10.0 ng/mL and 20.0 ng/mL, to be tested;

IV. Lipid Food Simulant Standard Working Solution Formulation

Weighing 2 g olive oil into six 10 mL stoppered test tubes accurately, and adding 5 μL, 10 μL, 20 μL, 50 μL, 100 μL, 200 μL of the mixed standard intermediate stock of Step II to obtain a mixed standard working solution with the content of 0.5 ng/mL, 1.0 ng/mL, 2.0 ng/mL, 5.0 ng/mL, 10.0 ng/mL and 20.0 ng/mL, adding 10 mL of methanol into each test tube respectively, vortexing for 2 min, and standing for layering, then sucking the upper solution by a syringe, filtering through a 0.2 μm hydrophobic polytetrafluoroethylene filter membrane, to be tested;

V. Migration Experiments

Referring to the migration test method and conditions of GB/T 23296.1-2009, selecting the simulant of aqueous food or lipid food to soak a sample;

VI. Sample Pretreatment Process

Diluting the aqueous food simulant with methanol for 10 times, mixing uniformly, and 1 mL of the diluted solution is aspirated by a glass syringe and filtered through a 0.22 μm PTFE syringe filter into a sample to be tested;

Weighing 2 g of olive oil food simulant into a 15 mL glass centrifuge tube with a plug, adding 5 mL of methanol, vortexing for 3 min, centrifuging for 5 min at 4000 r/min, removing the upper-layer methanol, repeatedly extracting the sample once with 5 mL of methanol, merging the upper-layer methanol, mixing uniformly, filtering into the sample through a 0.22 μm hydrophobic polytetrafluoroethylene needle filter, to be tested;

VII. Liquid Chromatography Conditions

Chromatographic column: shim-pack XR-ODS III (1.6 μm, 2.0 mm×75 mm), column temperature 40° C., mobile phase A water, mobile phase B methanol, flow rate 0.3 mL/min, injection volume 5 μL, elution gradient 0-8 min, 90% B-100% B; 8-12 min, 100% B; 12-13 min, 100% B-90% B; 13-15 min, 90% B;

VIII. Mass Spectrometry Conditions

Ion source: an electrospray ion source (ESI); electrospray voltage (IS): positive ion mode 5500V and negative ion mode −4500V; atomizing gas pressure (GS 1, kPa): 55; curtain Air Pressure (CUR, kPa): 35; auxiliary gas flow rate (GS 2, kPa): 55; ion source temperature (TEM): 600° C.; the detection method comprises the following steps: the multi-reaction detection (MRM), the collision voltage (CE), the de-clustering voltage (DP) and the collision cell exit voltage (CXP) of each substance are shown in Table 1.

### Embodiment 2

According to the method of embodiment 1, a specific amount of migration of 16 antioxidants in 20 parts PET/PE compound food packaging material samples, respectively. The results show that the above 16 species were not detected in 4% acetic acid and ultrapure water food analog (Table 3, Table 4); in a 10% ethanol analog, there was 14 parts of the sample detected Irganox 168, the content was in the range of 46 jig/kg to 826 μg/kg, 11 parts of the sample showed Irganox 1076, the content was in the range of 81 μg/kg to 525 μg/kg (Table 5), in the olive oil food analog, there were 4 samples detected Irganox 1076 in the range of 92 μg/kg to 120 μg/kg (Table 6). The amount of migration of the detected substance is lower than that of GB 9685-2016.

TABLE 3
4% acetic acid simulant

| | Anti- | Anti- | Anti- | Anti- | Anti- | Anti- | Anti- | Anti- | Anti- | Anti- | Anti- | Anti- | Anti- | Anti- | Anti- | |
|---|---|---|---|---|---|---|---|---|---|---|---|---|---|---|---|---|
| | oxi- | oxi- | oxi- | oxi- | oxi- | oxi- | oxi- | oxi- | oxi- | oxi- | oxi- | oxi- | oxi- | oxi- | oxi- | |
| | dant | dant | dant | dant | dant | dant | dant | dant | dant | dant | dant | dant | dant | dant | dant | |
| Number | DLTP | 425 | 168 | 405 | 3114 | 2246 | 300 | 697 | CA | 245 | 1290 | 1024 | CY | 1098 | 1076 | BHA |
| 1 | — | — | — | — | — | — | — | — | — | — | — | — | — | — | — | — |
| 2 | — | — | — | — | — | — | — | — | — | — | — | — | — | — | — | — |
| 3 | — | — | — | — | — | — | — | — | — | — | — | — | — | — | — | — |
| 4 | — | — | — | — | — | — | — | — | — | — | — | — | — | — | — | — |
| 5 | — | — | — | — | — | — | — | — | — | — | — | — | — | — | — | — |
| 6 | — | — | — | — | — | — | — | — | — | — | — | — | — | — | — | — |
| 7 | — | — | — | — | — | — | — | — | — | — | — | — | — | — | — | — |
| 8 | — | — | — | — | — | — | — | — | — | — | — | — | — | — | — | — |
| 9 | — | — | — | — | — | — | — | — | — | — | — | — | — | — | — | — |
| 10 | — | — | — | — | — | — | — | — | — | — | — | — | — | — | — | — |
| 11 | — | — | — | — | — | — | — | — | — | — | — | — | — | — | — | — |
| 12 | — | — | — | — | — | — | — | — | — | — | — | — | — | — | — | — |
| 13 | — | — | — | — | — | — | — | — | — | — | — | — | — | — | — | — |
| 14 | — | — | — | — | — | — | — | — | — | — | — | — | — | — | — | — |
| 15 | — | — | — | — | — | — | — | — | — | — | — | — | — | — | — | — |
| 16 | — | — | — | — | — | — | — | — | — | — | — | — | — | — | — | — |
| 17 | — | — | — | — | — | — | — | — | — | — | — | — | — | — | — | — |
| 18 | — | — | — | — | — | — | — | — | — | — | — | — | — | — | — | — |
| 19 | — | — | — | — | — | — | — | — | — | — | — | — | — | — | — | — |
| 20 | — | — | — | — | — | — | — | — | — | — | — | — | — | — | — | — |

Unit: μg/L

TABLE 4
ultrapure water simulant

| | Anti- | Anti- | Anti- | Anti- | Anti- | Anti- | Anti- | Anti- | Anti- | Anti- | Anti- | Anti- | Anti- | Anti- | Anti- | |
|---|---|---|---|---|---|---|---|---|---|---|---|---|---|---|---|---|
| | oxi- | oxi- | oxi- | oxi- | oxi- | oxi- | oxi- | oxi- | oxi- | oxi- | oxi- | oxi- | oxi- | oxi- | oxi- | |
| | dant | dant | dant | dant | dant | dant | dant | dant | dant | dant | dant | dant | dant | dant | dant | |
| Number | DLTP | 425 | 168 | 405 | 3114 | 2246 | 300 | 697 | CA | 245 | 1290 | 1024 | CY | 1098 | 1076 | BHA |
| 1 | — | — | — | — | — | — | — | — | — | — | — | — | — | — | — | — |
| 2 | — | — | — | — | — | — | — | — | — | — | — | — | — | — | — | — |
| 3 | — | — | — | — | — | — | — | — | — | — | — | — | — | — | — | — |
| 4 | — | — | — | — | — | — | — | — | — | — | — | — | — | — | — | — |
| 5 | — | — | — | — | — | — | — | — | — | — | — | — | — | — | — | — |
| 6 | — | — | — | — | — | — | — | — | — | — | — | — | — | — | — | — |
| 7 | — | — | — | — | — | — | — | — | — | — | — | — | — | — | — | — |
| 8 | — | — | — | — | — | — | — | — | — | — | — | — | — | — | — | — |
| 9 | — | — | — | — | — | — | — | — | — | — | — | — | — | — | — | — |
| 10 | — | — | — | — | — | — | — | — | — | — | — | — | — | — | — | — |
| 11 | — | — | — | — | — | — | — | — | — | — | — | — | — | — | — | — |
| 12 | — | — | — | — | — | — | — | — | — | — | — | — | — | — | — | — |
| 13 | — | — | — | — | — | — | — | — | — | — | — | — | — | — | — | — |
| 14 | — | — | — | — | — | — | — | — | — | — | — | — | — | — | — | — |
| 15 | — | — | — | — | — | — | — | — | — | — | — | — | — | — | — | — |
| 16 | — | — | — | — | — | — | — | — | — | — | — | — | — | — | — | — |
| 17 | — | — | — | — | — | — | — | — | — | — | — | — | — | — | — | — |
| 18 | — | — | — | — | — | — | — | — | — | — | — | — | — | — | — | — |
| 19 | — | — | — | — | — | — | — | — | — | — | — | — | — | — | — | — |
| 20 | — | — | — | — | — | — | — | — | — | — | — | — | — | — | — | — |

Unit: μg/L

TABLE 5
10% ethanol simulant

| | Anti- | Anti- | Anti- | Anti- | Anti- | Anti- | Anti- | Anti- | Anti- | Anti- | Anti- | Anti- | Anti- | Anti- | Anti- | |
|---|---|---|---|---|---|---|---|---|---|---|---|---|---|---|---|---|
| | oxi- | oxi- | oxi- | oxi- | oxi- | oxi- | oxi- | oxi- | oxi- | oxi- | oxi- | oxi- | oxi- | oxi- | oxi- | |
| | dant | dant | dant | dant | dant | dant | dant | dant | dant | dant | dant | dant | dant | dant | dant | |
| Number | DLTP | 425 | 168 | 405 | 3114 | 226 | 300 | 697 | CA | 245 | 1290 | 1024 | CY | 1098 | 1076 | BHA |
| 1 | — | — | 46 | — | — | — | — | — | — | — | — | — | — | — | — | — |
| 2 | — | — | 297 | — | — | — | — | — | — | — | — | — | — | — | — | — |
| 3 | — | — | 441 | — | — | — | — | — | — | — | — | — | — | — | 291 | — |
| 4 | — | — | 474 | — | — | — | — | — | — | — | — | — | — | — | — | — |
| 5 | — | — | 485 | — | — | — | — | — | — | — | — | — | — | — | 446 | — |
| 6 | — | — | 426 | — | — | — | — | — | — | — | — | — | — | — | 324 | — |
| 7 | — | — | 346 | — | — | — | — | — | — | — | — | — | — | — | 16 | — |
| 8 | — | — | 534 | — | — | — | — | — | — | — | — | — | — | — | 10 | — |
| 9 | — | — | 826 | — | — | — | — | — | — | — | — | — | — | — | 328 | — |
| 10 | — | — | 249 | — | — | — | — | — | — | — | — | — | — | — | 320 | — |
| 11 | — | — | 538 | — | — | — | — | — | — | — | — | — | — | — | 81 | — |
| 12 | — | — | 389 | — | — | — | — | — | — | — | — | — | — | — | 515 | — |
| 13 | — | — | 778 | — | — | — | — | — | — | — | — | — | — | — | 525 | — |
| 14 | — | — | 574 | — | — | — | — | — | — | — | — | — | — | — | 227 | — |
| 15 | — | — | — | — | — | — | — | — | — | — | — | — | — | — | — | — |
| 16 | — | — | — | — | — | — | — | — | — | — | — | — | — | — | — | — |
| 17 | — | — | — | — | — | — | — | — | — | — | — | — | — | — | — | — |
| 18 | — | — | — | — | — | — | — | — | — | — | — | — | — | — | — | — |
| 19 | — | — | — | — | — | — | — | — | — | — | — | — | — | — | — | — |
| 20 | — | — | — | — | — | — | — | — | — | — | — | — | — | — | — | — |

Unit: μg/L

TABLE 6
olive oil simulant

| | Anti- | Anti- | Anti- | Anti- | Anti- | Anti- | Anti- | Anti- | Anti- | Anti- | Anti- | Anti- | Anti- | Anti- | Anti- | |
|---|---|---|---|---|---|---|---|---|---|---|---|---|---|---|---|---|
| | oxi- | oxi- | oxi- | oxi- | oxi- | oxi- | oxi- | oxi- | oxi- | oxi- | oxi- | oxi- | oxi- | oxi- | oxi- | |
| | dant | dant | dant | dant | dant | dant | dant | dant | dant | dant | dant | dant | dant | dant | dant | |
| Number | DLTP | 425 | 168 | 405 | 3114 | 2246 | 300 | 697 | CA | 245 | 1290 | 1024 | CY | 1098 | 1076 | BHA |
| 1 | — | — | — | — | — | — | — | — | — | — | — | — | — | — | 120.00 | — |
| 2 | — | — | — | — | — | — | — | — | — | — | — | — | — | — | — | — |
| 3 | — | — | — | — | — | — | — | — | — | — | — | — | — | — | — | — |
| 4 | — | — | — | — | — | — | — | — | — | — | — | — | — | — | — | — |
| 5 | — | — | — | — | — | — | — | — | — | — | — | — | — | — | 115.00 | — |
| 6 | — | — | — | — | — | — | — | — | — | — | — | — | — | — | — | — |
| 7 | — | — | — | — | — | — | — | — | — | — | — | — | — | — | — | — |
| 8 | — | — | — | — | — | — | — | — | — | — | — | — | — | — | — | — |
| 9 | — | — | — | — | — | — | — | — | — | — | — | — | — | — | — | — |
| 10 | — | — | — | — | — | — | — | — | — | — | — | — | — | — | — | — |
| 11 | — | — | — | — | — | — | — | — | — | — | — | — | — | — | — | — |
| 12 | — | — | — | — | — | — | — | — | — | — | — | — | — | — | — | — |
| 13 | — | — | — | — | — | — | — | — | — | — | — | — | — | — | 141.00 | — |
| 14 | — | — | — | — | — | — | — | — | — | — | — | — | — | — | 92.00 | — |
| 15 | — | — | — | — | — | — | — | — | — | — | — | — | — | — | — | — |
| 16 | — | — | — | — | — | — | — | — | — | — | — | — | — | — | — | — |
| 17 | — | — | — | — | — | — | — | — | — | — | — | — | — | — | — | — |
| 18 | — | — | — | — | — | — | — | — | — | — | — | — | — | — | — | — |
| 19 | — | — | — | — | — | — | — | — | — | — | — | — | — | — | — | — |
| 20 | — | — | — | — | — | — | — | — | — | — | — | — | — | — | — | — |

Unit: μg/L

### Embodiment 1

Selection of Olive Oil Food Simulant Extraction Solvent

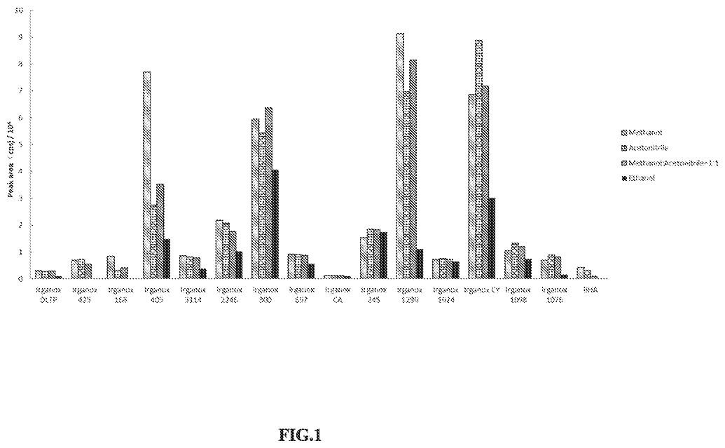

The extraction of antioxidants in olive oil with a content of 50 Ng/kg was compared to four solvents with methanol, acetonitrile, 50% methanol acetonitrile and ethanol (FIG. 1). Results show that the extraction effect of methanol, acetonitrile, 50% methanol acetonitrile and ethanol on antioxidant in olive oil is poor in sequence. The extraction effect of the ethanol against the oxidant is generally poor, and the extraction of the irganox irganox 425, irganox wen 168, bha is not possible; Acetonitrile has a better extraction effect on irganox cy, but the extraction effect on other compounds has no obvious advantage compared with other solvents; the 50% methanol acetonitrile has a good extraction effect on irganox 300, and the overall extraction effect is between methanol and acetonitrile; the extraction effect of methanol on Irganox 168, Irganox 405 and Irganox 1290 is better than the other three solvents, and the overall extraction effect is the best, so methanol is selected to extract the antioxidant in olive oil.

### Embodiment 2

Selection of extraction time for olive oil food simulant

The effect of adding 16 antioxidants with the same concentration in the olive oil food analog is extracted with methanol, the influence of different times (0.5, 1.0, 1.5, 2.0, 2.5, 3.0, 3.5, 4.0 min) on the extraction effect is investigated, the result shows that the chromatographic peak area of the 16 antioxidants is gradually increased from beginning to 2 mins, the extraction time is continuously prolonged, and the chromatographic peak area of the 16 antioxidants has no obvious change, so that the extraction time is selected to be 3 min.

### Embodiment 3

Optimization of Chromatographic Separation Conditions

The separation effect of ODS and phenyl both types of chromatographic columns against the oxidant was investigated, it was found that 16 compounds had better peak-type and suitable retention on both types of chromatographic columns, but the ODS-type chromatographic column was better than the phenyl column on the overall mass spectral response value, thus ultimately selecting the ODS-type chromatographic column.

When a methanol-water mobile phase system is adopted, 16 compounds can obtain better mass spectrum response values, and the peak separation degree of each compound is good, and the peak type no-front stretching or trailing phenomenon is obtained. When 0.1% formic acid is added in the aqueous phase, the response value of each compound is not significantly improved, and in the negative ion mode, formic acid has a severe inhibitory effect on the mass spectral response values of irganox 1076, bha, resulting in a peak-out.

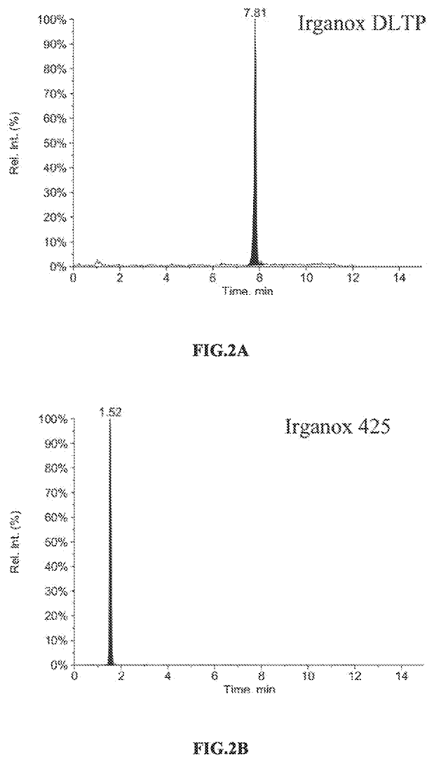

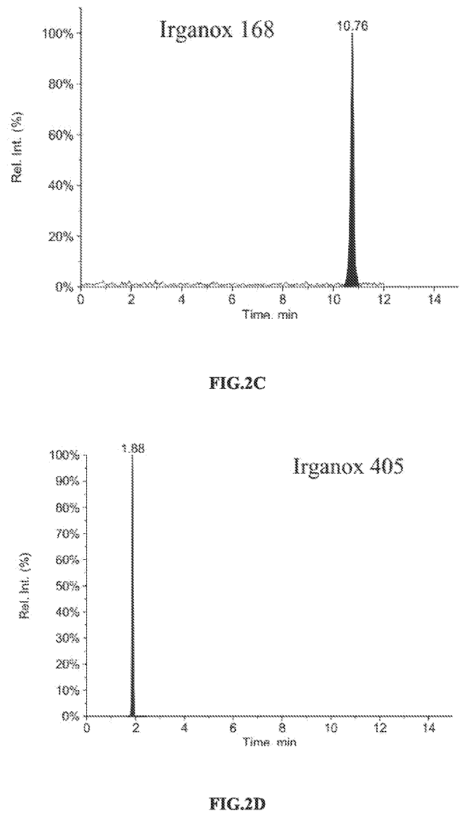

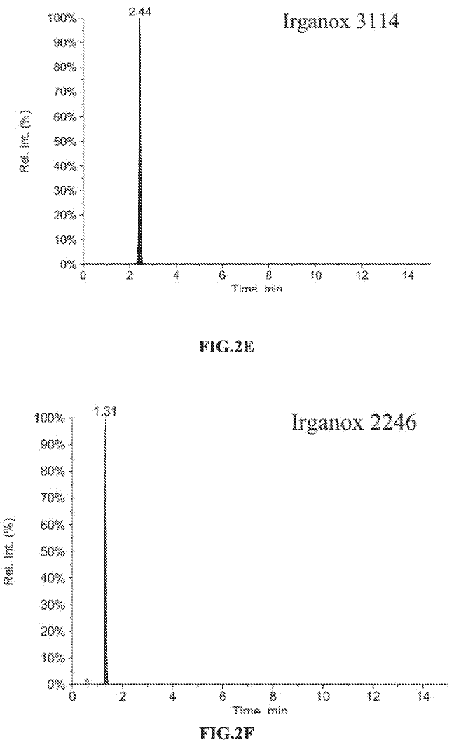

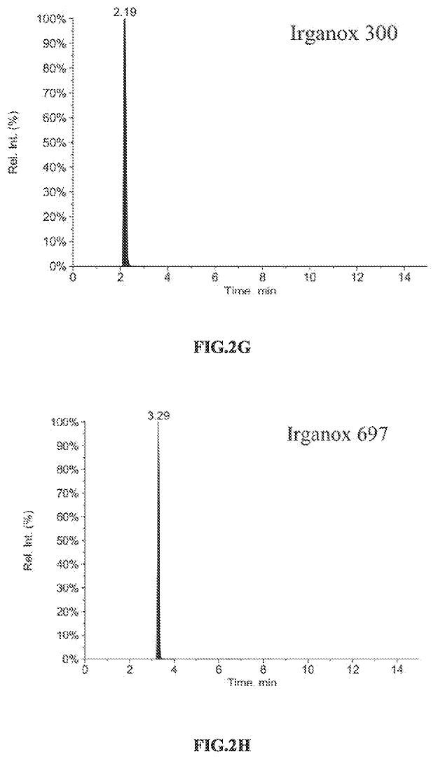

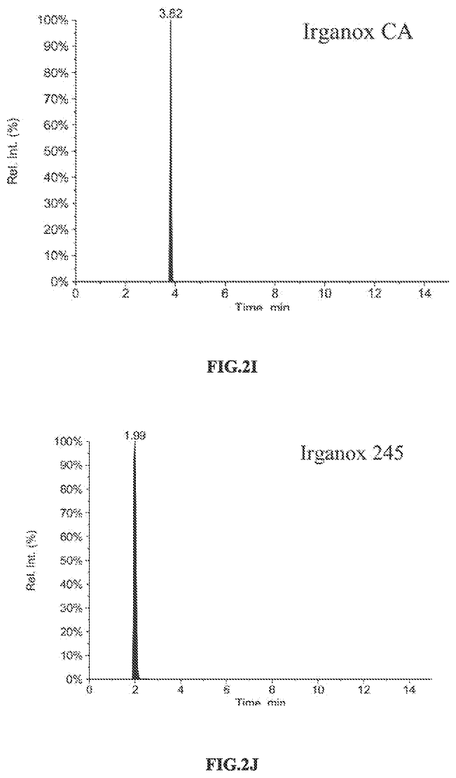

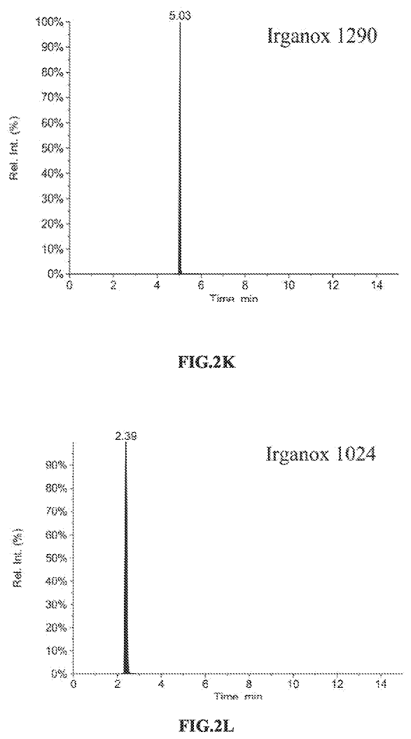

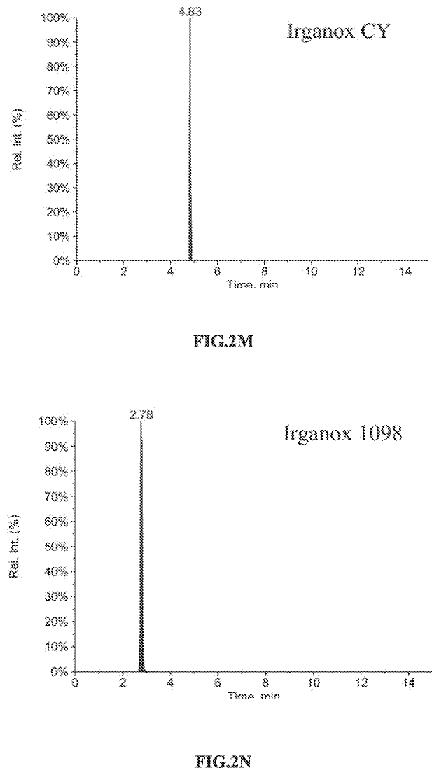

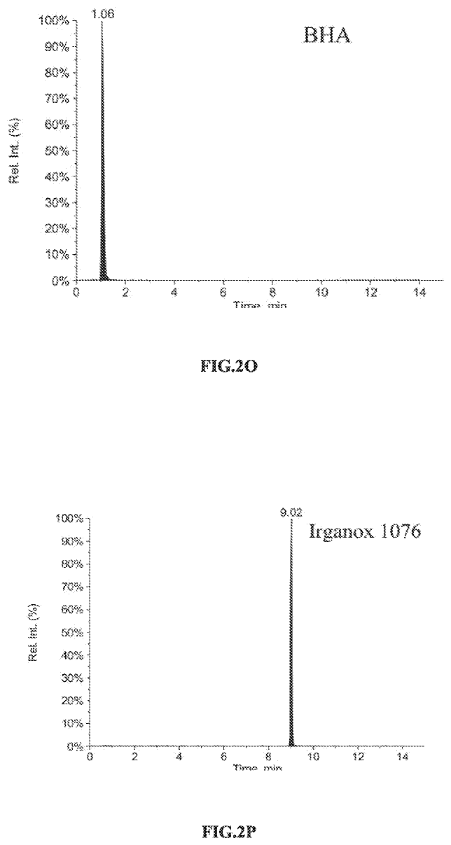

In comprehensive consideration, methanol-water was selected as the mobile phase for gradient elution with 16 antioxidants reaching baseline separation within 15 mins, wherein the extracted ion chromatogram in 10% ethanol food analog are shown in FIGS. 2A-2P

### Embodiment 4

Linear Linear Equations, Detection Limits, and Quantitation Limits for Methods of Detection

16 kinds of antioxidant mixed standard solutions were formulated with 10 times diluted aqueous food analog (ultrapure water, 4% acetic acid, 10% ethanol), respectively, at a concentration of 0.5 ng/mL, 1.0 ng/mL, 2.0 ng/mL, 5.0 ng/mL, 10 ng/mene, 20 ng/mmole;

A mixed standard working solution of 2.5 μg/kg, 5.0 μg/kg, 10 μg/kg, 25 μg/kg, 50 μg/kg, 100 μg/kg was formulated with olive oil simulant. The calibration curve is plotted with the concentration or the content of the abscissa (X) and the peak area of the chromatographic peak is the ordinate (Y). Results are shown in Table 7. In the range of 0.5-20 ng/ml and 2.5-100 μg/kg, the linear relationship of 16 antioxidants in four food simulants is good (r>0.995). The limit of quantitation (LOQ) of 16 antioxidants in water-based food simulants was calculated by diluting the standard solution with corresponding food simulants (olive oil food simulants with blank olive oil methanol extract) until the signal-to-noise ratio (s/N) was equal to 10. The LOQ of 16 antioxidants in aqueous food simulants was 0.1˜1.3 ng/ml, and the LOQ of olive oil food simulants was 0.3˜3.0 μg/kg.

TABLE 7

| | | Linear range | | | LOQ |
|---|---|---|---|---|---|
| Food | | (ng/mL, | Regression | | (ng/mL, |
| simulant | Compound | [μg/kg^{a}) | equation | r | μg/kg^{a}) |
| 10% | Irganox DLTP | 0.5~20 | y = 1.74 × 10⁵ x + 5.46 × 10⁴ | 0.9993 | 0.4 |
| Ethanol | Irganox 425 | 0.5~20 | y = 1.64 × 10⁵ x + 9.77 × 10⁴ | 0.9992 | 0.3 |
| 4% | Irganox 168 | 0.5~20 | y = 4.86 × 10⁴ x + 3.05 × 10⁴ | 0.9993 | 0.5 |
| Acetic | Irganox 405 | 0.5~20 | y = 1.36 × 10⁶ x + 1.58 × 10⁵ | 0.9994 | 0.3 |
| acid | Irganox 3114 | 0.5~20 | y = 1.8 × 10⁵ x − 2.18 × 10⁴ | 0.9996 | 0.5 |
| H₂O | Irganox 2246 | 0.5~20 | y = 1.68 × 10⁵ x + 6.03 × 10⁴ | 0.9993 | 0.3 |
| Olive oil | Irganox 300 | 0.5~20 | y = 4.47 × 10⁶ x + 1.88 × 10⁶ | 0.9991 | 0.4 |
| | Irganox 697 | 0.5~20 | y = 1.02 × 10⁵ x + 6.05 × 10³ | 0.9995 | 0.2 |
| | Irganox CA | 0.5~20 | y = 5.71 × 10³ x + 964 | 0.9999 | 1.2 |
| | Irganox 245 | 0.5~20 | y = 1.3 × 10⁶ x + 4.22 × 10⁴ | 0.9997 | 0.2 |
| | Irganox 1290 | 0.5~20 | y = 1.46 × 10⁶ x + 8.33 × 10⁵ | 0.9993 | 0.1 |
| | Irganox 1024 | 0.5~20 | y = 1.31 × 10⁵ x + 9.98 × 10³ | 0.9998 | 0.3 |
| | Irganox CY | 0.5~20 | y = 1.02 × 10⁶ x + 5.2 × 10⁵ | 0.9996 | 0.2 |
| | Irganox 1098 | 0.5~20 | y = 4.16 × 10⁵ x − 2.51 × 10⁴ | 0.9998 | 0.2 |
| | Irganox 1076 | 0.5~20 | y = 2.09 × 10⁴ x + 4.64 × 10³ | 0.9999 | 0.4 |
| | BHA | 0.5~20 | y = 8.29 × 10⁵ x − 2.06 × 10⁵ | 0.9996 | 0.4 |
| | Irganox DLTP | 0.5~20 | y = 1.82 × 10⁴ x + 1.2 × 10³ | 0.9991 | 0.4 |
| | Irganox 425 | 0.5~20 | y = 1.62 × 10⁵ x − 1.95 × 10⁴ | 1.0000 | 0.3 |
| | Irganox 168 | 0.5~20 | y = 5.43 × 10⁴ x − 1.38 × 10⁴ | 0.9993 | 0.4 |
| | Irganox 405 | 0.5~20 | y = 3.79 × 10⁵ x − 1.39 × 10⁵ | 0.9990 | 0.2 |
| | Irganox 3114 | 0.5~20 | y = 1.78 × 10⁵ x + 2.4 × 10⁴ | 0.9997 | 0.5 |
| | Irganox 2246 | 0.5~20 | y = 1.28 × 10⁵ x − 3.13 × 10⁴ | 0.9990 | 0.3 |
| | Irganox 300 | 0.5~20 | y = 5.9 × 10⁵ x − 6.79 × 10⁴ | 1.0000 | 0.2 |
| | Irganox 697 | 0.5~20 | y = 8.87 × 10⁴ x − 7.37 × 10³ | 0.9998 | 0.5 |
| | Irganox CA | 0.5~20 | y = 6.8 × 10³ x − 852 | 0.9991 | 1.3 |
| | Irganox 245 | 0.5~20 | y = 1.56 × 10⁵ x − 3.22 × 10⁴ | 0.9994 | 0.5 |
| | Irganox 1290 | 0.5~20 | y = 2.51 × 10⁵ x + 1.33 × 10⁴ | 0.9999 | 0.1 |
| | Irganox 1024 | 0.5~20 | y = 6.38 × 10⁴ x − 3.06 × 10³ | 0.9999 | 0.4 |
| | Irganox CY | 0.5~20 | y = 5.08 × 10⁵ x + 2.26 × 10⁵ | 0.9996 | 0.1 |
| | Irganox 1098 | 0.5~20 | y = 1.08 × 10⁵ x − 6.25 × 10³ | 0.9998 | 0.3 |
| | Irganox 1076 | 0.5~20 | y = 1.14 × 10⁴ x + 7.43 × 10³ | 0.9990 | 0.4 |
| | BHA | 0.5~20 | y = 5.51 × 10⁴ x − 3.45 × 10³ | 0.9998 | 0.5 |
| | Irganox DLTP | 0.5~20 | y = 1.59 × 10⁴ x + 1.53 × 10³ | 0.9999 | 0 5 |
| | Irganox 425 | 0.5~20 | y = 1.72 × 10⁵ x − 9.26 × 10³ | 0.9997 | 0.2 |
| | Irganox 168 | 0.5~20 | y = 2.95 × 10⁴ x + 8.63 × 10⁴ | 0.9993 | 0.2 |
| | Irganox 405 | 0.5~20 | y = 1.22 × 10⁶ x − 1.91 × 10⁵ | 0.9997 | 0.1 |
| | Irganox 3114 | 0.5~20 | y = 1.42 × 10⁵ x + 2.6 × 10⁴ | 0.9996 | 0.5 |
| | Irganox 2246 | 0.5~20 | y = 1.45 × 10⁵ x − 1.56 × 10⁴ | 0.9997 | 0.2 |
| | Irganox 300 | 0.5~20 | y = 5.8 × 10⁵ x − 5.34 × 10⁴ | 0.9998 | 0.1 |
| | Irganox 697 | 0.5~20 | y = 9.29 × 10⁴ x − 3.72 × 10³ | 0.9998 | 0.2 |
| | Irganox CA | 0.5~20 | y = 6.44 × 10³ x − 389 | 0.9991 | 1.0 |
| | Irganox 245 | 0.5~20 | y = 1.63 × 10⁵ x − 2.89 × 10⁴ | 0.9995 | 0.6 |
| | Irganox 1290 | 0.5~20 | y = 2.56 × 10⁵ x − 4.5 × 10⁴ | 0.9996 | 0.1 |
| | Irganox 1024 | 0.5~20 | y = 6.14 × 10⁴ x − 6.87 × 10³ | 0.9995 | 0.5 |
| | Irganox CY | 0.5~20 | y = 5.31 × 10⁵ x + 8.74 × 10⁴ | 0.9995 | 0.1 |
| | Irganox 1098 | 0.5~20 | y = 1.12 × 10⁵ x + 9.46 × 10³ | 0.9998 | 0.2 |
| | Irganox 1076 | 0.5~20 | y = 3.59 × 10⁴ x + 6.04 × 10³ | 0.9998 | 0.5 |
| | BHA | 0.5~20 | y = 1.25 × 10⁵ x − 3.33 × 10⁴ | 0.9995 | 0.5 |
| | Irganox DLTP | 2.5~100 | y = 1.55 × 10⁴ x + 5.91 × 10³ | 0.9985 | 3.0 |
| | Irganox 425 | 2.5~100 | y = 4.66 × 10⁴ x + 1.39 × 10⁴ | 0.9993 | 1.4 |
| | Irganox 168 | 2.5~100 | y = 1.68 × 10⁴ x + 1.5 × 10³ | 0.9981 | 1.5 |
| | Irganox 405 | 2.5~100 | y = 9.99 × 10⁵ x − 4.1 × 10⁵ | 0.9997 | 0.4 |
| | Irganox 3114 | 2.5~100 | y = 4.14 × 10³ x + 5.82 × 10³ | 0.9991 | 2.5 |
| | Irganox 2246 | 2.5~100 | y = 1.1 × 10⁴ x + 4.99 × 10³ | 0.9996 | 1.6 |
| | Irganox 300 | 2.5~100 | y = 5.99 × 10⁵ x − 2.46 × 10⁵ | 0.9993 | 1.0 |
| | Irganox 697 | 2.5~100 | y = 9.23 × 10⁴ x − 3.36 × 10⁴ | 0.9995 | 2.0 |
| | Irganox CA | 2.5~100 | y = 1.21 × 10⁴ x + 2.53 × 10³ | 0.9985 | 2.0 |
| | Irganox 245 | 2.5~100 | y = 1.7 × 10⁵ x − 9.05 × 10⁴ | 0.9992 | 0.8 |
| | Irganox 1290 | 2.5~100 | y = 6.17 × 10⁵ x + 5.55 × 10⁵ | 0.9991 | 0.3 |
| | Irganox 1024 | 2.5~100 | y = 7.7 × 10⁴ x − 3.03 × 10⁴ | 0.9997 | 0.6 |
| | Irganox CY | 2.5~100 | y = 7.23 × 10⁵ x − 1.76 × 10⁵ | 0.9994 | 0.5 |
| | Irganox 1098 | 2.5~100 | y = 1.22 × 10⁵ x − 7.14 × 10⁴ | 0.9982 | 1.0 |
| | Irganox 1076 | 2.5~100 | y = 2.84 × 10⁵ x − 6.69 × 10⁴ | 0.9994 | 1.2 |
| | BHA | 2.5~100 | y = 3.9 × 10⁴ x − 9.85 × 10³ | 0.9997 | 1.5 |

### Embodiment 5

Recovery and Precision of Detection Method

A negative PET/PE compound food contact material sample is selected, three levels of labeling recovery experiments (N=6) are respectively used for four different food simulators according to the experimental conditions, the average recovery rate of 16 antioxidants at 2.0 to 20 μg/kg plus scale is 81.0% to 112%, the relative standard deviation (RSD) is 0.4% to 9.1%, and the results are shown in Table 8.

TABLE 8

| | | | 10% | | 4% | | | | | |
|---|---|---|---|---|---|---|---|---|---|---|
| | | | Ethanol | | Acetic Acid | | H₂O | | Olive oil | |
| | | | Re- | | Re- | | Re- | | Re- | |
| | Com- | Spiked | covery | RSD | covery | RSD | covery | RSD | covery | RSD |
| No. | pound | μg/kg | % | % | % | % | % | % | % | % |
| 1 | Irganox | 2.0 | 91.2 | 1.2 | 87.5 | 8.7 | 89.2 | 5.2 | 86.5 | 7.4 |
| | DLTP | 5.0 | 95.2 | 0.6 | 88.9 | 3.5 | 94.2 | 6.2 | 84.2 | 0.7 |
| | | 20.0 | 90.7 | 7.8 | 93.8 | 6.0 | 95.8 | 6.6 | 88.7 | 2.6 |
| 2 | Irganox | 2.0 | 96.3 | 4.7 | 97.9 | 6.6 | 92.1 | 5.6 | 90.9 | 0.6 |
| | 425 | 5.0 | 94.3 | 4.0 | 92.6 | 5.4 | 89.1 | 7.3 | 85.7 | 8.4 |
| | | 20.0 | 101 | 8.4 | 98.4 | 4.6 | 102 | 8.7 | 92.8 | 3.6 |
| 3 | Irganox | 2.0 | 90.3 | 3.8 | 92.5 | 7.0 | 98.7 | 6.2 | 88.6 | 7.1 |
| | 168 | 5.0 | 87.6 | 3.8 | 91.2 | 5.7 | 94.3 | 3.1 | 83.5 | 4.7 |
| | | 20.0 | 88.7 | 5.8 | 95.6 | 5.4 | 101 | 2.3 | 89.8 | 4.7 |
| 4 | Irganox | 2.0 | 89.0 | 5.5 | 90.2 | 5.0 | 92.0 | 5.8 | 91.1 | 7.0 |
| | 405 | 5.0 | 92.1 | 0.7 | 93.3 | 4.6 | 97.0 | 7.3 | 92.8 | 0.4 |
| | | 20.0 | 95.7 | 5.6 | 91.9 | 5.8 | 105 | 1.6 | 88.9 | 0.8 |
| 5 | Irganox | 2.0 | 82.1 | 4.3 | 89.4 | 0.7 | 90.3 | 3.3 | 106 | 2.4 |
| | 3114 | 5.0 | 85.8 | 8.4 | 88.0 | 3.7 | 87.6 | 9.1 | 104 | 8.8 |
| | | 20.0 | 86.2 | 1.3 | 92.1 | 4.6 | 94.5 | 9.0 | 97.2 | 6.6 |
| 6 | Irganox | 2.0 | 81.9 | 1.5 | 88.5 | 6.0 | 101 | 5.6 | 85.4 | 5.8 |
| | 2246 | 5.0 | 86.4 | 9.1 | 90.6 | 8.8 | 112 | 2.6 | 87.7 | 8.7 |
| | | 20.0 | 86.7 | 3.4 | 96.4 | 8.7 | 96.8 | 6.6 | 87.0 | 1.9 |
| 7 | Irganox | 2.0 | 88.6 | 6.1 | 89.7 | 7.3 | 92.7 | 7.7 | 90.3 | 5.7 |
| | 300 | 5.0 | 95.3 | 2.9 | 90.7 | 3.1 | 98.2 | 5.3 | 93.3 | 1.5 |
| | | 20.0 | 96.5 | 8.2 | 94.1 | 4.0 | 92.2 | 8.8 | 92.7 | 6.1 |
| 8 | Irganox | 2.0 | 96.9 | 5.5 | 90.6 | 6.1 | 94.9 | 6.8 | 94.1 | 6.5 |
| | 697 | 5.0 | 112 | 7.7 | 101 | 0.4 | 99.2 | 8.1 | 95.0 | 2.2 |
| | | 20.0 | 106 | 4.3 | 104 | 1.7 | 101 | 0.7 | 102 | 8.5 |
| 9 | Irganox | 2.0 | 88.9 | 0.7 | 87.3 | 5.4 | 88.1 | 3.1 | 85.8 | 1.1 |
| | CA | 5.0 | 102 | 8.1 | 97.6 | 0.9 | 102 | 4.3 | 107 | 0.6 |
| | | 20.0 | 110 | 3.8 | 97.0 | 3.9 | 90.9 | 0.9 | 105 | 6.3 |
| 10 | Irganox | 2.0 | 92.6 | 7.4 | 86.5 | 1.1 | 90.4 | 5.8 | 86.3 | 1.7 |
| | 245 | 5.0 | 94.6 | 6.9 | 88.0 | 9.0 | 96.4 | 8.6 | 87.9 | 8.4 |
| | | 20.0 | 97.7 | 5.9 | 84.7 | 6.3 | 97.5 | 5.4 | 92.4 | 3.4 |
| 11 | Irganox | 2.0 | 95.5 | 5.7 | 82.1 | 8.6 | 102 | 1.6 | 84.1 | 1.9 |
| | 1290 | 5.0 | 103 | 5.0 | 83.9 | 9.0 | 96.3 | 7.5 | 87.9 | 3.5 |
| | | 20.0 | 97.1 | 3.5 | 86.8 | 8.7 | 89.5 | 6.6 | 90.8 | 7.5 |
| 12 | Irganox | 2.0 | 90.0 | 7.9 | 101 | 8.2 | 97.4 | 7.5 | 94.4 | 8.9 |
| | 1024 | 5.0 | 104 | 3.4 | 94.6 | 7.7 | 105 | 4.7 | 97.9 | 0.6 |
| | | 20.0 | 87.6 | 8.4 | 97.3 | 3.6 | 108 | 8.7 | 103 | 3.0 |
| 13 | Irganox | 2.0 | 81.0 | 3.4 | 93.2 | 8.7 | 109 | 7.5 | 103 | 3.7 |
| | CY | 5.0 | 83.4 | 0.7 | 96.9 | 5.1 | 97.8 | 7.4 | 92.9 | 4.0 |
| | | 20.0 | 92.1 | 4.0 | 97.0 | 5.5 | 110 | 3.1 | 93.4 | 4.3 |
| 14 | Irganox | 2.0 | 92.1 | 3.6 | 105 | 6.3 | 97.2 | 4.0 | 86.0 | 5.4 |
| | 1098 | 5.0 | 93.5 | 8.3 | 96.3 | 1.0 | 108 | 4.6 | 106 | 4.4 |
| | | 20.0 | 97.7 | 2.3 | 96.9 | 8.4 | 106 | 4.4 | 94.0 | 2.4 |
| 15 | Irganox | 2.0 | 90.5 | 7.0 | 93.0 | 7.0 | 97.9 | 4.3 | 97.0 | 7.7 |
| | 1076 | 5.0 | 92.9 | 8.8 | 84.5 | 8.8 | 94.5 | 5.1 | 90.8 | 8.4 |
| | | 20.0 | 89.9 | 2.5 | 86.8 | 9.1 | 102 | 3.5 | 87.6 | 1.9 |
| 16 | BHA | 2.0 | 88.0 | 2.3 | 110 | 0.5 | 90.2 | 8.0 | 89.8 | 1.6 |
| | | 5.0 | 96.5 | 7.5 | 95.7 | 7.3 | 94.8 | 7.0 | 86.0 | 3.0 |
| | | 20.0 | 98.2 | 5.3 | 98.6 | 0.5 | 98.2 | 1.1 | 87.9 | 2.7 |

The previous description of the disclosed embodiments is provided to enable any person skilled in the art to make or use the disclosure. Various modifications to these embodiments will be readily apparent to those skilled in the art, and the generic principles defined herein may be practiced in other embodiments without departing from the spirit or scope of the disclosure. Thus, the present disclosure will not be limited to the embodiments shown herein but is to be accorded the widest scope consistent with the principles and novel features disclosed herein.

What is claimed is:

1. A method for measuring specific migration amount of an antioxidant in a polyethylene terephthalate/polyethylene compound food contact material, comprising:
I. standard stock solution formulation
weighing an antioxidant standard substance precisely and dissolving the antioxidant standard substance in methanol for constant volume to prepare a mixed standard stock solution with a concentration of 100 μg/mL, and storing the mixed standard stock solution under −20° C.;
II. mixed standard intermediate stock solution formulation
accurately sucking 1 mL of the mixed standard stock solution in the step I in a 100 mL volumetric flask respectively, and carrying out constant volume by methanol to obtain a mixed standard intermediate stock solution with the concentration of 1 μg/mL;
III. aqueous food simulant standard working solution formulation
transferring 5 μL, 10 μL, 20 μL, 50 μL, 100 μL, 200 μL of the mixed standard intermediate stock solution obtained in the step II into six 10-mL volumetric flasks respectively, carrying out volumetric calibration on the mixed standard intermediate stock solution by using an aqueous food simulant diluted by 10 times of methanol, and mixing uniformly to obtain a mixed standard working solution with the concentration of 0.5 ng/mL, 1.0 ng/mL, 2.0 ng/mL, 5.0 ng/mL, 10.0 ng/mL and 20.0 ng/mL to be tested;
IV. lipid food simulant standard working solution formulation
weighing 2 g olive oil into six 10 mL stoppered test tubes accurately, and adding 5 μL, 10 μL, 20 μL, 50 μL, 100 μL, 200 μL of the mixed standard intermediate stock in the step II to obtain a mixed standard working solution with the content of 0.5 ng/mL, 1.0 ng/mL, 2.0 ng/mL, 5.0 ng/mL, 10.0 ng/mL and 20.0 ng/mL, adding 10 mL of methanol into each test tube respectively, vortexing for 2 min, and standing for layering, then sucking the upper solution by a syringe, filtering through a 0.2 μm hydrophobic polytetrafluoroethylene filter membrane to be tested;
V. migration experiments
selecting the simulant of aqueous food or lipid food to soak a sample by referring to the migration test method and conditions of GB/T 23296.1-2009;
VI. sample pretreatment process
diluting the aqueous food simulant with methanol for 10 times, mixing uniformly, and 1 mL of the diluted solution is aspirated by a glass syringe and filtered through a 0.22 μm PTFE syringe filter into a sample to be tested;
weighing 2 g of olive oil food simulant into a 15 mL glass centrifuge tube with a plug, adding 5 mL of methanol, vortexing for 3 min, centrifuging for 5 min at 4000 r/min, removing the upper-layer methanol, repeatedly extracting the sample once with 5 mL of methanol, merging the upper-layer methanol, mixing uniformly, filtering into the sample through a 0.22 μm hydrophobic polytetrafluoroethylene needle filter, to be tested;
VII. liquid chromatography conditions
chromatographic column: shim-pack XR-ODSIII (1.6 μm, 2.0 mm×75 mm), column temperature 40° C., mobile phase A water, mobile phase B methanol, flow rate 0.3 mL/min, injection volume 5 μL, elution gradient 0-8 min, 90% B-100% B; 8-12 min, 100% B; 12-13 min, 100% B-90% B; 13-15 min, 90% B;
VIII. mass spectrometry conditions
an electrospray ion source, wherein the electrospray voltage is 5500V in a positive ion mode, 4500V in a negative ion mode; the atomization air pressure is 55 kPa, the air curtain air pressure is 35 kPa, the auxiliary air flow rate is 55 kPa, the ion source temperature is 600° C., the scanning mode is positive ion scanning and negative ion scanning, and the detection method is multi-reaction detection.

2. The method for measuring specific migration amount of an antioxidant in a polyethylene terephthalate/polyethylene compound food contact material of claim 1, wherein the the antioxidant standard substance in step I comprises Irganox DLTP, Irganox 425, Irganox 168, Irganox 405, Irganox 3114, I One or more of rganox 2246, Irganox 300, Irganox 697, Irganox CA, Irganox 245, Irganox 1290, Irganox 1024, Irganox CY, Irganox 1098, Irganox 1076, or BHA.

3. The method for measuring specific migration amount of an antioxidant in a polyethylene terephthalate/polyethylene compound food contact material of claim 2, wherein the mass spectrometry conditions further comprise a collision voltage (CE), a de-clustering voltage (DP), and a collision cell exit voltage (CXP) for each antioxidant; and the CE, the DP and the CXP for each antioxidant are shown in following table;

| com- | | | | | | |
|---|---|---|---|---|---|---|
| pound | com- | ion pair | | DP | CE | CXP |
| No. | pound | (m/z) | polarity | (eV) | (eV) | (eV) |
| 1 | IRGANOX | 515.4 > 143.1* | positive | 200 | 23 | 16 |
| | DLTP | 515.4 > 115.0 | | | 35 | 12 |
| 2 | IRGANOX | 386.3 > 191.1* | positive | 60 | 23 | 17 |
| | 425 | 386.3 > 257.1 | | | 15 | 37 |
| 3 | IRGANOX | 647.5 > 347.4* | positive | 120 | 47 | 13 |
| | 168 | 647.5 > 147.1 | | | 47 | 13 |
| 4 | IRGANOX | 406.3 > 196.2* | positive | 100 | 48 | 12 |
| | 405 | 406.3 > 119.1 | | | 41 | 11 |
| 5 | IRGANOX | 806.3 > 219.0* | positive | 100 | 72 | 13 |
| | 3114 | 806.3 > 370.3 | | | 53 | 24 |
| 6 | IRGANOX | 358.2 > 177.1* | positive | 50 | 22 | 10 |
| | 2246 | 358.2 > 229.2 | | | 13 | 16 |
| 7 | IRGANOX | 357.1 > 194.1* | negative | −100 | −39 | −19 |
| | 300 | 357.1 > 179.2 | | | −59 | −11 |
| 8 | IRGANOX | 695.5 > 277.1* | negative | −200 | −46 | −8 |
| | 697 | 695.5 > 417.2 | | | −34 | −33 |
| 9 | IRGANOX | 543.4 > 337.1* | negative | −230 | −62 | −14 |
| | CA | 543.4 > 281.1 | | | −54 | −23 |
| 10 | IRGANOX | 585.4 > 367.1* | negative | −100 | −39 | −23 |
| | 245 | 585.4 > 409.1 | | | −36 | −28 |
| 11 | IRGANOX | 438.3 > 205.2* | negative | −50 | −42 | −10 |
| | 1290 | 438.3 > 232.2 | | | −36 | −20 |
| 12 | IRGANOX | 551.4 > 333.4* | negative | −200 | −38 | −21 |
| | 1024 | 551.4 > 115.1 | | | −42 | −6 |
| 13 | IRGANOX | 698.5 > 508.2* | negative | −100 | −38 | −13 |
| | CY | 698.5 > 232.0 | | | −58 | −18 |
| 14 | IRGANOX | 635.6 > 417.2* | negative | −100 | −52 | −20 |
| | 1098 | 635.6 > 199.1 | | | −57 | −14 |
| 15 | IRGANOX | 529.2 > 267.2* | negative | −100 | −51 | −24 |
| | 1076 | 529.2 > 269.2 | | | −39 | −23 |
| 16 | BHA | 179.1 > 149.0* | negative | −50 | −31 | −15 |
| | | 179.1 > 164.1 | | | −20 | −10. |

4. The method for measuring specific migration amount of an antioxidant in a polyethylene terephthalate/polyethylene compound food contact material of claim 1, wherein the aqueous food simulant comprises ultrapure water, 4% acetic acid, or 10% ethanol.
